(19) United States
(12) Patent Application Publication
(10) Pub. No.: US 2013/0143196 A1
(43) Pub. Date: Jun. 6, 2013

(54) METHODS AND MATERIALS FOR PROLONGING USEFUL STORAGE OF RED BLOOD CELL PREPARATIONS AND PLATELET PREPARATIONS

(71) Applicants: Michael J. Joyner, Rochester, MN (US); Daniel G. Ericson, Rochester, MN (US)

(72) Inventors: Michael J. Joyner, Rochester, MN (US); Daniel G. Ericson, Rochester, MN (US)

(73) Assignees: Dynasil Biomedical Corporation, Watertown, MA (US); Mayo Foundation for Medical Education and Research, Rochester, MN (US)

(21) Appl. No.: 13/816,775

(22) PCT Filed: Mar. 16, 2012
(86) PCT No.: PCT/US12/29510

§ 371 Date: Feb. 13, 2013

Related U.S. Application Data
(60) Provisional application No. 61/453,455, filed on Mar. 16, 2011.
(60) Provisional application No. 61/532,331, filed on Sep. 8, 2011.

(51) Int. Cl. C12N 5/078

(52) U.S. Cl. CPC C12N 5/0641
USPC 435/2; 422/547

(57) ABSTRACT

This document provides methods and materials for enhancing the storage capabilities of red blood cell preparations. For example, methods and materials for using CO₂ to store red blood cells in a manner that (a) reduces the level of glucose or 2,3-DPG consumption of or reduces the level of 2,3-DPG production by a red blood cell preparation, (b) reduces the level of lactate formation by a red blood cell preparation, and/or (c) reduces the pH level of a red blood cell preparation are provided. Such methods and materials can result in prolonging the useful lifespan of the red blood cells of the red blood cell preparation. This document also provides methods and materials involved in prolonging useful storage of platelet preparations. For example, methods and materials for storing platelets in a manner that reduces platelet metabolism, that preserves platelet function, and/or that reduces the risk of bacterial contamination are provided.

### BACKGROUND

1. Technical Field

This document relates to methods and materials involved in prolonging useful storage of red blood cell preparations. For example, this document relates to methods and materials for storing red blood cells in a manner that reduces the level of glucose or 2,3-diphosphoglycerate (2,3-DPG) consumption of or reduces the level of 2,3 DPG production by a red blood cell preparation, that reduces the level of lactate formation by a red blood cell preparation, and/or that reduces the pH level of a red blood cell preparation, thereby prolonging the useful lifespan of the red blood cells of the red blood cell preparation. This document also relates to methods and materials involved in prolonging useful storage of platelet preparations. For example, this document relates to methods and materials for storing platelets in a manner that reduces platelet metabolism, that preserves platelet function, and/or that reduces the risk of bacterial contamination, thereby prolonging the useful lifespan of the platelets of a platelet preparation.

2. Background Information

In general, red blood cells can be stored for about 42 days in blood banks During this time, a so-called storage lesion can occur, thereby limiting the ability of the surviving red blood cells to carry oxygen. Storage lesions are thought to result from anaerobic glycolysis, depleted energy stores in the cells, a reduction in 2-3 DPG, and other adverse metabolic events including oxidative stress.

There is an increase in demand for platelet transfusions in clinical medicine. Meeting the demand for platelets is challenging because they can have a limited shelf life. The short shelf life also makes the efficient collection, processing, and distribution of platelets challenging.

### SUMMARY

This document provides methods and materials for enhancing the storage capabilities of red blood cell preparations. For example, this document provides methods and materials for using CO₂ to store red blood cells in a manner that (a) reduces the level of glucose or 2,3-DPG consumption of or reduces the level of 2,3 DPG production by a red blood cell preparation, (b) reduces the level of lactate formation by a red blood cell preparation, and/or (c) reduces the pH level of a red blood cell preparation. Such methods and materials can result in prolonging the useful lifespan of the red blood cells of the red blood cell preparation.

In general, storage of red blood cells (RBCs) at 4° C. can result in an increase in the oxygen affinity of the RBC hemoglobin (Hb), which can have negative consequences for the delivery of oxygen to tissue after transfusion. During storage of RBCs, large amounts of lactate can be formed, and blood pH can drop rapidly. Because glycolysis is temperature-dependent and pH-dependent, the more time passes between collection and refrigeration, the more rapid the reduction in pH will be. 2,3-DPG levels also can drop quickly when stored at room temperature. When the pH falls below about 7.2 (e.g., below 7.2 to 7.3), the bisphosphoglycerate phosphatase will be activated, and the normally high concentration of 2,3-DPG is rapidly depleted.

As described herein, red blood cells stored in an environment (e.g., a bag) containing CO₂ (e.g., greater than about 100 mmHg of pCO₂, greater than about 200 mmHg of pCO₂, greater than about 300 mmHg of pCO₂, greater than about 400 mmHg of pCO₂, or greater than about 500 mmHg of pCO₂) can exhibit reduced glucose consumption, reduced lactate formation, and lower pH levels than red blood cells stored under normal environmental air conditions (e.g., air having about 21 percent O₂ and about 40 to 60 mmHg of pCO₂). The drop in pH can occur as soon as the blood is exposed to CO₂, but this drop in pH is reversible. In general, it is not favorable to drop pH below 6.3, but the methods and materials provided here can be used to reduce pH in a manner that is reversible. In some cases, 100 percent CO₂, a mixture of CO₂ and Nitrogen (e.g., 50/50 CO₂—N), or a mixture of CO₂ and air (e.g., 50/50 CO₂-air) can be used as described herein, for example, to lower pH levels.

Red blood cells exposed to CO₂ and exhibiting reduced glucose consumption, reduced lactate formation, and lower pH levels (e.g., rapidly reversible lower pH levels) can have a longer useful lifespan than red blood cells not exhibiting reduced glucose consumption, reduced lactate formation, and lower pH levels. In some cases, the lower pH levels observed for red blood cells stored under conditions of greater than about 100 mmHg of pCO₂ (e.g., pH levels less than about 6.6, less than about 6.5, less than about 6.4, less than about 6.3, or less than about 6.2) can be reversible such that red blood cells switched from CO₂ conditions of greater than about 100 mmHg of pCO₂ to normal air conditions (e.g., about 21 percent O₂ and about 40 to 60 mmHg of pCO₂) can exhibit pH levels greater than about 6.6 (e.g., greater than about 6.7, greater than about 6.8, greater than about 6.9, or greater than about 7.0).

This document also provides methods and materials for enhancing the storage capabilities of platelet preparations. For example, this document provides methods and materials for using CO₂ to store platelets in a manner that (a) reduces platelet metabolism, (b) preserves platelet function, and/or (c) reduces the risk of bacterial contamination. Such methods and materials can result in prolonging the useful lifespan of the platelets of a platelet preparation.

Since platelets stored at temperatures from about 2° C. to about 6° C. typically form platelet aggregates, platelets are generally stored in high glucose media and at room temperature to reduce formation of platelet aggregates. Platelets stored in high glucose media and at room temperature, however, can deteriorate over time and have an increased risk of undergoing bacterial contamination. As described herein, a platelet preparation stored in an environment (e.g., a bag) containing CO₂ (e.g., greater than about 100 mmHg of pCO₂, greater than about 200 mmHg of pCO₂, greater than about 300 mmHg of pCO₂, greater than about 400 mmHg of pCO₂, or greater than about 500 mmHg of pCO₂) can exhibit reduced platelet metabolism, preserved platelet function, and reduced bacterial growth when compared to platelet preparations stored under normal environmental air conditions (e.g., air having about 21 percent O₂ and about 40 to 60 mmHg of pCO₂). In some cases, a platelet preparation stored in an environment (e.g., a bag) containing CO₂ (e.g., greater than about 100 mmHg of pCO₂, greater than about 200 mmHg of pCO₂, greater than about 300 mmHg of pCO₂, greater than about 400 mmHg of pCO₂, or greater than about 500 mmHg of pCO₂) can be stored at a temperature from about 2° C. to about 6° C. without forming a significant amount or a detectable amount of platelet aggregates.

In some cases, a drop in pH can occur as soon as a platelet preparation is exposed to CO₂, but this drop in pH can be reversible. In general, it is not favorable to drop pH below 6.0, but the methods and materials provided herein can be used to reduce pH in a manner that is reversible.

In some cases, a blood or platelet container (e.g., a blood collection bag or a platelet collection bag) provided herein can include an entry port for inserting blood or platelets into the blood or platelet container and a capsule positioned in a manner to deliver CO₂ material to the inner region of the blood or platelet container. For example, a blood or platelet container provided herein can include a breakable capsule located within the inner region of the blood or platelet container. The breakable capsule can house any type of material designed to deliver CO₂ gas to the inner region of the blood or platelet container. For example, the capsule can house CO₂ gas or a powder that produces CO₂ gas (e.g., bicarbonate). Before or after adding a red blood cell preparation (e.g., blood) to the blood container or before or after adding a platelet preparation to a platelet container, the capsule can be pierced or broken to release CO₂ gas into the inner region of the blood container or platelet container.

In some cases, a blood container (e.g., blood collection bag) provided herein can include an entry port for inserting blood into the blood container and can be connected to one or more satellite containers (e.g., a satellite bag) designed to house CO₂ gas or material capable of generating CO₂ gas. Before or after adding a red blood cell preparation (e.g., blood) to the blood container, a satellite container can be manipulated to move CO₂ gas into the blood container, thereby allowing CO₂ to contact the red blood cell preparation to be stored.

In some cases, a blood container (e.g., blood collection bag) provided herein can include an entry port for inserting blood into the blood container and an injection port configured to allow sterile CO₂ gas or material capable of generating CO₂ gas to be injected into the blood container. Before or after adding a red blood cell preparation (e.g., blood) to the blood container, sterile CO₂ gas or material capable of generating CO₂ gas can be injected into the blood container, thereby allowing CO₂ to contact the red blood cell preparation to be stored.

In some cases, a platelet container (e.g., platelet collection bag) provided herein can include an entry port for inserting platelets into the platelet container and can be connected to one or more satellite containers (e.g., a satellite bag) designed to house CO₂ gas or material capable of generating CO₂ gas. Before or after adding a platelet preparation (e.g., platelets) to the platelet container, a satellite container can be manipulated to move CO₂ gas into the platelet container, thereby allowing CO₂ to contact the platelet preparation to be stored.

In some cases, a platelet container (e.g., platelet collection bag) provided herein can include an entry port for inserting platelets into the platelet container and an injection port configured to allow sterile CO₂ gas or material capable of generating CO₂ gas to be injected into the platelet container. Before or after adding a platelet preparation (e.g., platelets) to the platelet container, sterile CO₂ gas or material capable of generating CO₂ gas can be injected into the platelet container, thereby allowing CO₂ to contact the platelet preparation to be stored.

In some cases, a platelet bag can be exposed to CO₂ in a CO₂ rich chamber, such as a bag within a bag. In such cases, the pH of the platelet preparation can drop to about 6.2 in about 10 minutes when, for example, commercially available platelet storage bags that have olefins within the plasticizer (e.g., Fenwal PL732 gas permable bags) are used to allow for gas exchange.

In general, one aspect of this document features a method for reducing the rate of lactate formation or glucose consumption by stored red blood cells of a red blood cell preparation. The method comprises, or consists essentially of, exposing the red blood cell preparation to CO₂ gas under conditions wherein the pCO₂ level of the red blood cell preparation is greater than 100 mmHg of pCO₂. The method can comprise exposing the red blood cell preparation to CO₂ gas under conditions wherein the pCO₂ level of the red blood cell preparation is greater than 200 mmHg of pCO₂. The method can comprise exposing the red blood cell preparation to CO₂ gas under conditions wherein the pCO₂ level of the red blood cell preparation is greater than 300 mmHg of pCO₂. The method can comprise exposing the red blood cell preparation to CO₂ gas under conditions wherein the pCO₂ level of the red blood cell preparation is greater than 400 mmHg of pCO₂. The method can comprise exposing the red blood cell preparation to CO₂ gas under conditions wherein the pCO₂ level of the red blood cell preparation is greater than 500 mmHg of pCO₂. The method can comprise exposing the red blood cell preparation to CO₂ gas under conditions wherein the pCO₂ level of the red blood cell preparation is between 200 and 600 mmHg of pCO₂. The method can comprise exposing the red blood cell preparation to CO₂ gas under conditions wherein the pCO₂ level of the red blood cell preparation is between 450 and 550 mmHg of pCO₂. The method can comprise reducing the rate of lactate formation to a level that results in less than 7.5 μmmol/mL of lactate being formed after 14 days of storage. The method can comprise reducing the rate of lactate formation to a level that results in less than 10 μmmol/mL of lactate being formed after 21 days of storage. The method can comprise reducing the rate of glucose consumption to a level that results in greater than 450 mg/dL of glucose being present after 14 days of storage. The method can comprise reducing the rate of glucose consumption to a level that results in greater than 200 mg/dL of glucose being present after 21 days of storage. The method can comprise reducing the rate of glucose consumption to a level that results in greater than 300 mg/dL of glucose being present after 21 days of storage. The method can comprise reducing the rate of glucose consumption to a level that results in greater than 400 mg/dL of glucose being present after 21 days of storage. The pH of the red blood cell preparation can be less than 6.8 after 14 days of storage. The pH of the red blood cell preparation can be less than 6.6 after 14 days of storage. The pH of the red blood cell preparation can be less than 6.4 after 14 days of storage. The pH of the red blood cell preparation can be less than 6.6 after 21 days of storage. The pH of the red blood cell preparation can be less than 6.4 after 21 days of storage.

In another aspect, this document features a method for reducing the rate of lactate formation or glucose consumption by stored red blood cells of a red blood cell preparation, wherein the method comprises, or consists essentially of, exposing the red blood cell preparation to CO₂ gas under conditions wherein the pH of the red blood cell preparation is less than 6.6. The method can comprise exposing the red blood cell preparation to CO₂ gas under conditions wherein the pH of the red blood cell preparation is less than 6.4. The method can comprise exposing the red blood cell preparation to CO₂ gas under conditions wherein the pH of the red blood cell preparation is less than 6.3. The pCO₂ of the red blood cell preparation can be greater than 100 mmHg of pCO₂. The pCO₂ of the red blood cell preparation can be greater than 200 mmHg of pCO₂. The pCO₂ of the red blood cell preparation can be greater than 300 mmHg of pCO₂. The pCO₂ of the red blood cell preparation can be greater than 400 mmHg of pCO₂. The pCO₂ of the red blood cell preparation can be greater than 500 mmHg of pCO₂. The pCO₂ of the red blood cell preparation can be between 200 and 600 mmHg of pCO₂. The pCO₂ of the red blood cell preparation can be between 450 and 550 mmHg of pCO₂. The method can comprise reducing the rate of lactate formation to a level that results in less than 7.5 μmol/mL of lactate being formed after 14 days of storage. The method can comprise reducing the rate of lactate formation to a level that results in less than 10 mmol/mL of lactate being formed after 21 days of storage. The method can comprise reducing the rate of glucose consumption to a level that results in greater than 450 mg/dL of glucose being present after 14 days of storage. The method can comprise reducing the rate of glucose consumption to a level that results in greater than 200 mg/dL of glucose being present after 21 days of storage. The method can comprise reducing the rate of glucose consumption to a level that results in greater than 300 mg/dL of glucose being present after 21 days of storage. The method can comprise reducing the rate of glucose consumption to a level that results in greater than 400 mg/dL of glucose being present after 21 days of storage.

In another aspect, this document features a blood container comprising, or consisting essentially of, an inlet port configured to allow blood to be inserted into the blood container and a capsule comprising CO₂ gas or material capable of generating CO₂ gas, wherein the capsule is located in a position that allows the CO₂ gas or the material to be delivered to an inner region of the container. The capsule can be located within the inner region of the container. The capsule can comprise CO₂ gas. The capsule can comprise material capable of generating CO₂ gas. The material can be a bicarbonate salt. The blood container can comprise an exhaust valve configured to allow gas to be removed from the inner region of the blood container.

In another aspect, this document features a blood container system comprising, or consisting essentially of, a blood container having an inlet port configured to allow blood to be inserted into the blood container and a satellite container comprising CO₂ gas or material capable of generating CO₂ gas, wherein the satellite container is configured to be in fluid communication with an inner region of the blood container. The system can comprise a valve configured to retain the CO₂ gas or material within the satellite container. The valve can be capable of being opened to allow the CO₂ gas or material to exit the satellite container into the inner region of the blood container. The system can comprise a membrane configured to retain the CO₂ gas or material within the satellite container. The membrane can be capable of being broken to allow the CO₂ gas or material to exit the satellite container into the inner region of the blood container. The blood container can comprise an exhaust valve configured to allow gas to be removed from the inner region of the blood container.

In another aspect, this document features a blood container comprising, or consisting essentially of, an inlet port configured to allow blood to be inserted into the blood container and an injection port configured to allow sterile insertion of a needle for delivering CO₂ gas or material capable of generating CO₂ gas into an inner region of the container. The blood container can comprise an exhaust valve configured to allow gas to be removed from the inner region of the blood container.

In another aspect, this document features a platelet container comprising, or consisting essentially of, an inlet port configured to allow platelets to be inserted into the platelet container and a capsule comprising CO₂ gas or material capable of generating CO₂ gas, wherein the capsule is located in a position that allows the CO₂ gas or the material to be delivered to an inner region of the container.

In another aspect, this document features a platelet container system comprising, or consisting essentially of, a platelet container having an inlet port configured to allow platelets to be inserted into the platelet container and a satellite container comprising CO₂ gas or material capable of generating CO₂ gas, wherein the satellite container is configured to be in fluid communication with an inner region of the platelet container.

In another aspect, this document features a platelet container comprising, or consisting essentially of, an inlet port configured to allow platelets to be inserted into the platelet container and an injection port configured to allow sterile insertion of a needle for delivering CO₂ gas or material capable of generating CO₂ gas into an inner region of the container.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a front view of one example of a blood container device containing a capsule having CO₂ gas or material capable of generating CO₂ gas.

FIG. 2 is a front view of an exemplary blood container system including a blood container device fluidly attached to multiple satellite containers.

FIG. 3 is a front view of an exemplary blood container system including a blood container device fluidly attached to one satellite container.

FIG. 4 is a front view of one example of a blood container device having an injection port for sterilely injecting CO₂ gas or material capable of generating CO₂ gas into an inner region of the blood container device.

FIG. 5 is a front view of an exemplary blood container device having an exhaust port for allowing gas to be removed from the inner region of the blood container device.

FIG. 6 is a front view of one example of a platelet container device containing a capsule having CO₂ gas or material capable of generating CO₂ gas.

FIG. 7 is a front view of an exemplary platelet container system including a platelet container device fluidly attached to multiple satellite containers.

FIG. 8 is a front view of an exemplary platelet container system including a platelet container device fluidly attached to one satellite container.

FIG. 9 is a front view of one example of a platelet container device having an injection port for sterilely injecting CO₂ gas or material capable of generating CO₂ gas into an inner region of the platelet container device.

FIG. 10 is a front view of an exemplary platelet container device having an exhaust port for allowing gas to be removed from the inner region of the platelet container device.

FIG. 11 is a graph plotting percent aggregation.

FIG. 12 is a graph plotting percent aggregation after reversing pH of CO₂ and control samples.

FIG. 13 is a graph plotting percent aggregation after 24 hours.

FIG. 14 is a front view of one example of a container having the ability to house one or more other smaller containers.

FIG. 15 is a front view of an inner region of a cabinet having the ability to house one or more containers.

FIG. 16 is a front view of a container and a cartridge for injecting CO₂ or other gas into the container.

FIG. 17 is a graph plotting bacterial growth (CFU/mL) of the indicated bacteria 48 hour after inoculation within either a preparation of control platelets or a preparation of platelets exposed to 99% CO₂ gas.

FIG. 18 is a bar graph plotting percent aggregation of platelets in PRP that were kept in room air (C) or in 99% CO₂ for 10 minutes or 24 hours. The 10 minute samples were assayed immediately or after they had re-attained pH 7.2. The 24-hour samples were assayed after the pH increased to 7.2.

### DETAILED DESCRIPTION

This document provides methods and materials for enhancing the storage capabilities of red blood cell preparations. For example, this document provides methods and materials for using CO₂ to store red blood cells in a manner that (a) reduces the level of glucose or 2,3-DPG consumption of or reduces the level of 2,3 DPG production by a red blood cell preparation, (b) reduces the level of lactate formation by a red blood cell preparation, and/or (c) reduces the pH level of a red blood cell preparation. Such methods and materials can result in prolonging the useful lifespan of the red blood cells of the red blood cell preparation.

As described herein, red blood cell preparations can be stored in an environment (e.g., a bag) containing CO₂ or can be exposed to CO₂ such that the level of pCO₂ of the preparation is greater than about 100 mmHg of pCO₂ (e.g., greater than about 150 mmHg of pCO₂, greater than about 200 mmHg of pCO₂, greater than about 300 mmHg of pCO₂, greater than about 400 mmHg of pCO₂, or greater than about 500 mmHg of pCO₂). In some cases, red blood cell preparations can be stored in an environment containing CO₂ or can be exposed to CO₂ such that the level of pCO₂ of the preparation is between about 100 mmHg of CO₂ and about 600 mmHg of pCO₂ (e.g., between about 150 mmHg of CO₂ and about 600 mmHg of pCO₂, between about 200 mmHg of CO₂ and about 600 mmHg of pCO₂, between about 200 mmHg of CO₂ and about 550 mmHg of pCO₂, or between about 450 mmHg of CO₂ and about 550 mmHg of pCO₂). In some cases, a minimum volume of CO₂ required to raise the pCO₂ of a 300 mL bag containing a red blood cell preparation to 400 mm Hg can be used. For example, 2, 3, 4, 5, 6, 7, 8, 9, 10, or more cubic centimeters of CO₂ can be used.

Any appropriate method can be used to expose a red blood cell preparation to CO₂. For example, CO₂ gas or material capable of generating CO₂ gas can be placed into a container designed to house a red blood cell preparation prior to adding the red blood cell preparation. Examples of materials capable of generating CO₂ gas include, without limitation, bicarbonate, bicarbonate salts, and bismuth subsalicylate. In some cases, CO₂ gas or material capable of generating CO₂ gas can be added to container already containing a red blood cell preparation. In some cases, excess gas can be removed from a container containing a red blood cell preparation exposed to CO₂ gas.

In some cases, a container that is not CO₂ permeable can be used to store a red blood cell preparation. For example, blood bags and glass containers can be used to store red blood cell preparations in a manner that maintains high CO₂ levels for at least 30, 40, 41, 42, 43, 45, 50, 55, or 60 days.

This document also provides methods and materials for enhancing the storage capabilities of platelet preparations. For example, this document provides methods and materials for using CO₂ to store platelets in a manner that (a) reduces platelet metabolism, (b) preserves platelet function, and/or (c) reduces the risk of bacterial contamination. Such methods and materials can result in prolonging the useful lifespan of the platelets of a platelet preparation.

As described herein, platelet preparations can be stored in an environment (e.g., a bag) containing CO₂ or can be exposed to CO₂ such that the level of pCO₂ of the preparation is greater than about 100 mmHg of pCO₂ (e.g., greater than about 150 mmHg of pCO₂, greater than about 200 mmHg of pCO₂, greater than about 300 mmHg of pCO₂, greater than about 400 mmHg of pCO₂, or greater than about 500 mmHg of pCO₂). In some cases, platelet preparations can be stored in an environment containing CO₂ or can be exposed to CO₂ such that the level of pCO₂ of the preparation is between about 100 mmHg of CO₂ and about 600 mmHg of pCO₂ (e.g., between about 150 mmHg of CO₂ and about 600 mmHg of pCO₂, between about 200 mmHg of CO₂ and about 600 mmHg of pCO₂, between about 200 mmHg of CO₂ and about 550 mmHg of pCO₂, or between about 450 mmHg of CO₂ and about 550 mmHg of pCO₂). In some cases, a minimum volume of CO₂ required to raise the pCO₂ of a 300 mL bag containing a platelet preparation to 400 mm Hg can be used. For example, 2, 3, 4, 5, 6, 7, 8, 9, 10, or more cubic centimeters of CO₂ can be used.

Any appropriate method can be used to expose a platelet preparation to CO₂. For example, CO₂ gas or material capable of generating CO₂ gas can be placed into a container designed to house a platelet preparation prior to adding the platelet preparation. Examples of materials capable of generating CO₂ gas include, without limitation, bicarbonate and bicarbonate salts. In some cases, CO₂ gas or material capable of generating CO₂ gas can be added to container already containing a platelet preparation. In some cases, excess gas can be removed from a container containing a platelet preparation exposed to CO₂ gas.

In some cases, a container that is not CO₂ permeable can be used to store a platelet preparation. For example, platelet bags and glass containers can be used to store platelet preparations in a manner that maintains high CO₂ levels for an appropriate period of time (e.g., until the product is released for transfusion). In another example, rigid PET bottles or glass bottles that do not have CO₂ exchange can be used. In some cases, at the time of release, a quick and easy de-gas step can be performed.

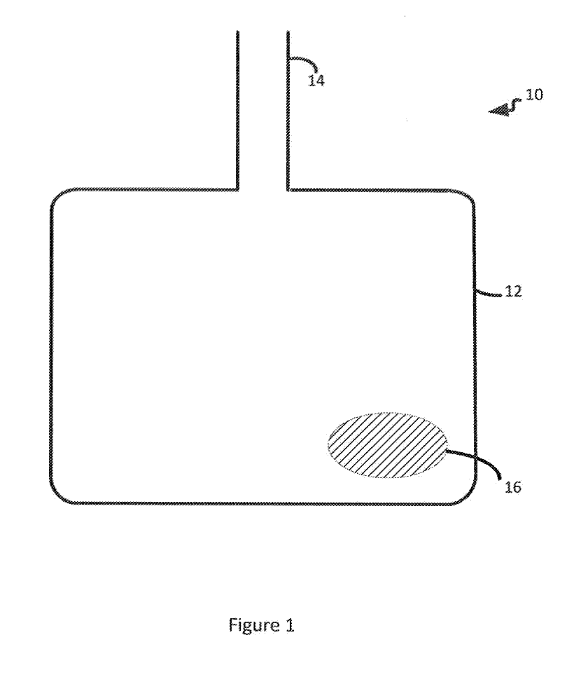

With reference to FIG. 1, blood container device 10 can include a blood container component 12 and an inlet port 14 configured to allow a red blood cell preparation to be inserted into blood container component 12. Blood container device 10 can include a capsule 16 within blood container component 12. Capsule 16 can contain CO₂ gas or material capable of generating CO₂ gas. In some cases, capsule 16 can be breakable such that a user can break capsule 16 (e.g., by squeezing) at a desired time point to release its contents (e.g., CO₂ gas or material capable of generating CO₂ gas).

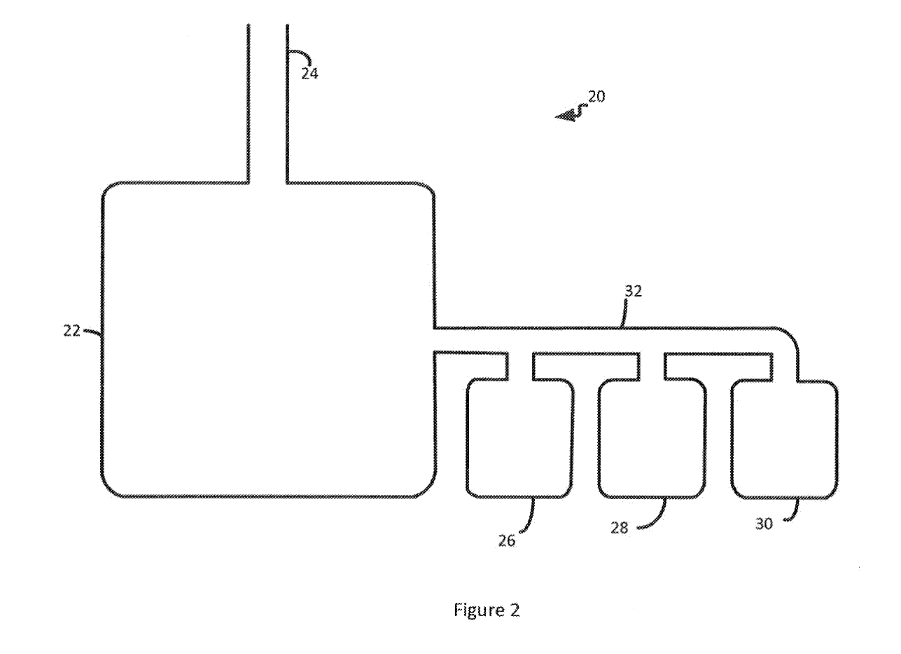

With reference to FIG. 2, blood container system 20 can include a blood container component 22 and an inlet port 24 configured to allow a red blood cell preparation to be inserted into blood container component 22. Blood container system 20 can include one or more satellite containers such as satellite containers 26, 28, and 30. Satellite containers 26, 28, and 30 can be fluidly connected to an inner region of blood container component 22 via a channel 32 (e.g., a tube). In some cases, satellite containers 26, 28, and 30 can be fluidly connected to each other. One or more of the satellite containers can contain CO₂ gas or material capable of generating CO₂ gas. For example, satellite container 30 can contain CO₂ gas or material capable of generating CO₂ gas. In some cases, blood container system 20 can include a valve or membrane configured to retain CO₂ gas or material capable of generating CO₂ gas within a satellite container until a user decides to allow the CO₂ gas or material capable of generating CO₂ gas to be released and moved into an inner region of blood container component 22.

In some cases, a satellite container can be designed to house blood components such as platelets or plasma. For example, satellite container 26 can be designed to house platelets, and satellite container 28 can be designed to house plasma.

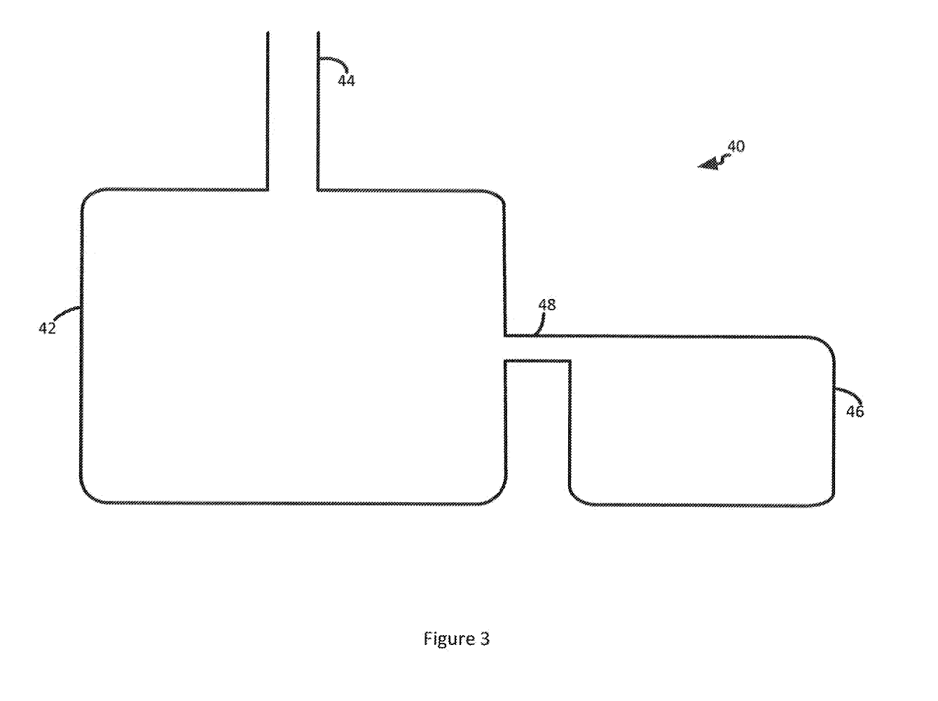

With reference to FIG. 3, blood container system 40 can include a blood container component 42 and an inlet port 44 configured to allow a red blood cell preparation to be inserted into blood container component 42. Blood container system 40 can include one satellite container (e.g., satellite container 46). Satellite container 46 can be fluidly connected to an inner region of blood container component 42 via a channel 48 (e.g., a tube). Satellite container 46 can contain CO₂ gas or material capable of generating CO₂ gas. In some cases, blood container system 40 can include a valve or membrane configured to retain CO₂ gas or material capable of generating CO₂ gas within satellite container 46 until a user decides to allow the CO₂ gas or material capable of generating CO₂ gas to be released and moved into an inner region of blood container component 42.

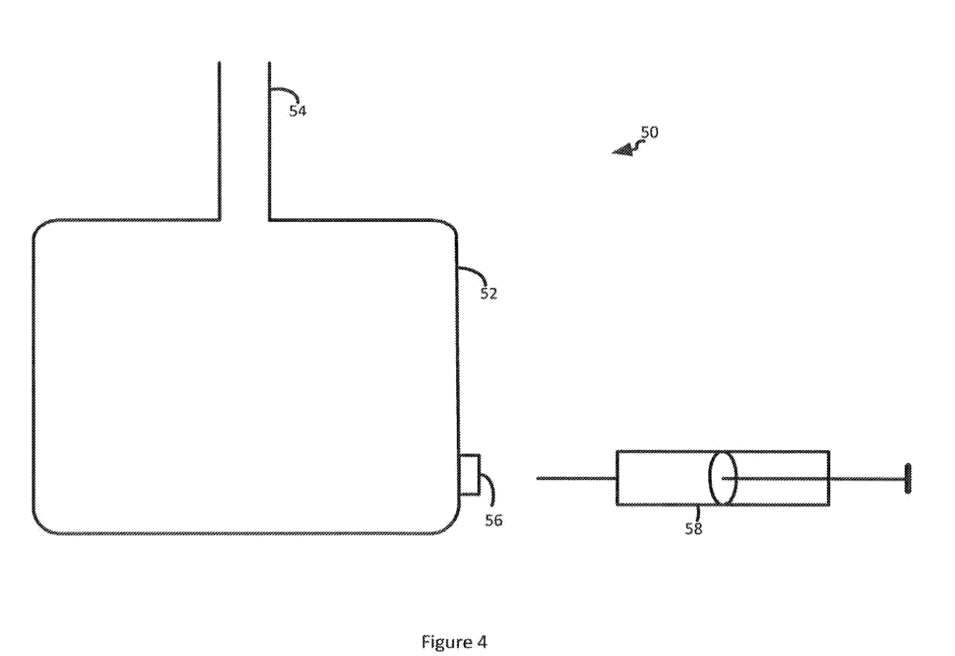

In some cases, a blood container component provided herein can include an injection port. For example, with reference to FIG. 4, a blood container device 50 can include a blood container component 52 and an inlet port 54 configured to allow a red blood cell preparation to be inserted into blood container component 52. Blood container device 50 can include an injection port 56. Injection port 56 can be configured to allow a needle (e.g., of a syringe 58) to be inserted into an inner region of blood container component 52. The syringe can be used to deliver CO₂ gas or material capable of generating CO₂ gas into an inner region of blood container component 52 in a sterile manner. In some cases, injection port 56 can be configured to seal upon removal of an inserted needle.

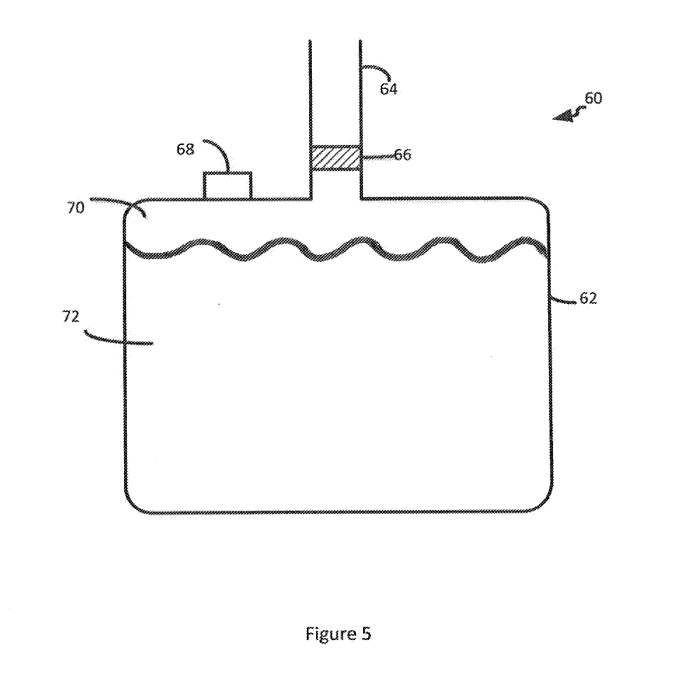

In some cases, a blood container component provided herein can include an exhaust valve. For example, with reference to FIG. 5, a blood container device 60 can include a blood container component 62 and an inlet port 64 configured to allow a red blood cell preparation 72 to be inserted into blood container component 62. Blood container device 60 can include an exhaust valve 68. Exhaust valve 68 can be configured to allow a user to remove gas 70 present within an inner region of blood container component 62 in a sterile and sealable manner.

In some cases, a blood container component provided herein can include an inlet port 64 having a valve. For example, with reference to FIG. 5, inlet port 64 can be configured to have valve 66. Valve 66 can be configured to have open and closed configurations. When in an open configuration, valve 66 can allow fluids and gases to pass into and out of an inner region of blood container component 62. When in a closed configuration, valve 66 can prevent the contents within an inner region of blood container component 62 from exiting that region via inlet port 64.

In some cases, a blood bag can include a series of bags to facilitate the separation of blood into components. The blood can be first collected as whole blood (e.g., typically around 500 cubic centimeters) in a large bag, and other bags can receive the separated products during processing. In some cases, one of the bags not used in the initial collection of whole blood can be loaded with CO₂ for subsequent evacuation and mixing with the red blood cells. In some cases, a relatively small amount (e.g., 50-100 cubic centimeters) of CO₂ can be used to saturate the red blood cells completely.

In some cases, a bubble-like wrap-like reservoir with CO₂ can be incorporated into the blood bag, and a simple one-way valve can be configured to permit evacuation of CO₂ with gentle squeezing into the remaining red blood cells. In some cases, a sterile one-way valve can be used to permit the rapid dosing of CO₂ from a traditional gas tank or other source. This can be configured in a manner that injects a known amount of CO₂ in the bag. Due to the nature of gas in closed spaces and equilibration characteristics of CO₂ and blood, minimal mixing may be needed. In some cases, dry ice or another chemical reservoir that includes a phase change from solid to gas can be placed in the blood bag to liberate CO₂.

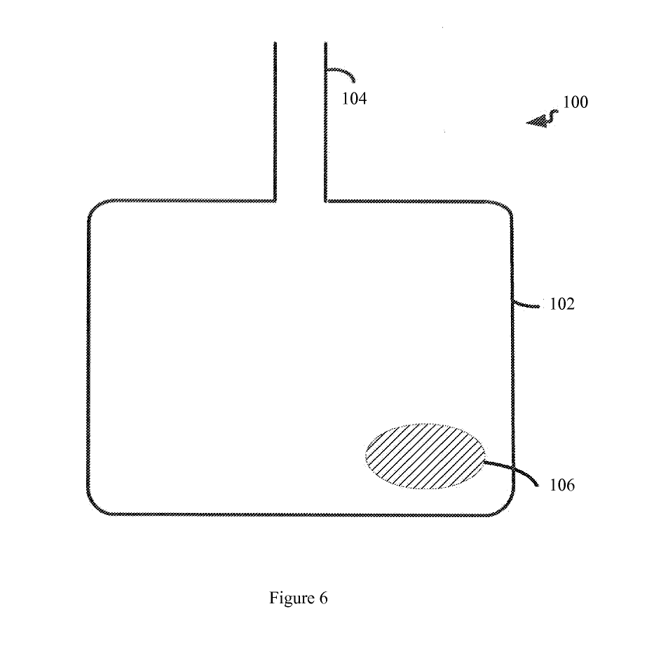

In some cases, a platelet container device similar to a blood container device provided herein can be used to house a platelet preparation. With reference to FIG. 6, platelet container device 100 can include a platelet container component 102 and an inlet port 104 configured to allow a platelet preparation to be inserted into platelet container component 102. Platelet container device 100 can include a capsule 106 within platelet container component 102. Capsule 106 can contain CO₂ gas or material capable of generating CO₂ gas. In some cases, capsule 106 can be breakable such that a user can break capsule 106 (e.g., by squeezing) at a desired time point to release its contents (e.g., CO₂ gas or material capable of generating CO₂ gas).

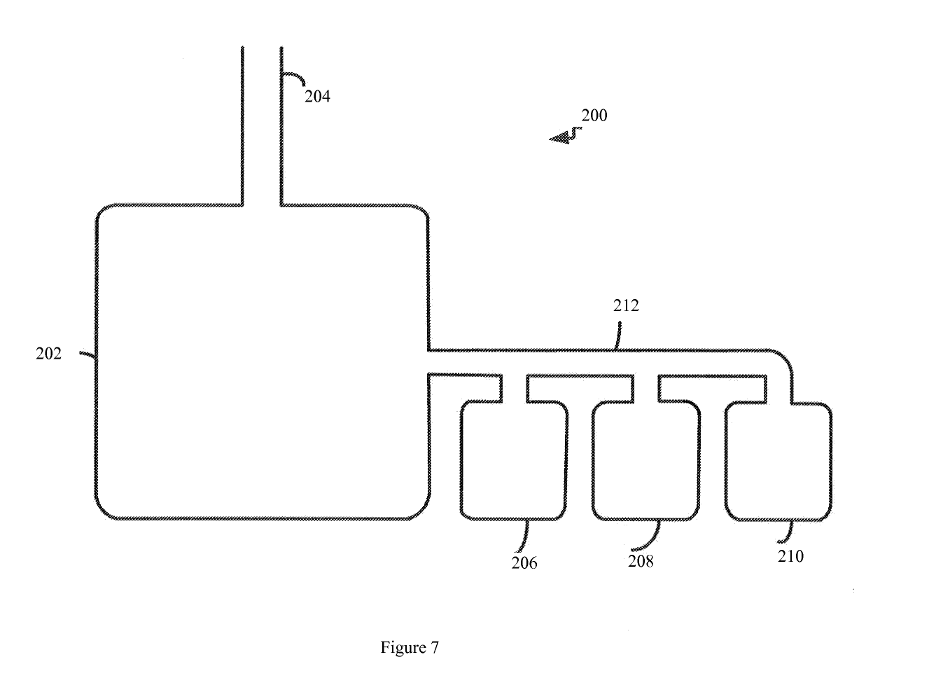

With reference to FIG. 7, platelet container system 200 can include a platelet container component 202 and an inlet port 204 configured to allow a platelet preparation to be inserted into platelet container component 202. Platelet container system 200 can include one or more satellite containers such as satellite containers 206, 208, and 210. Satellite containers 206, 208, and 210 can be fluidly connected to an inner region of platelet container component 202 via a channel 212 (e.g., a tube). In some cases, satellite containers 206, 208, and 210 can be fluidly connected to each other. One or more of the satellite containers can contain CO₂ gas or material capable of generating CO₂ gas. For example, satellite container 210 can contain CO₂ gas or material capable of generating CO₂ gas. In some cases, platelet container system 200 can include a valve or membrane configured to retain CO₂ gas or material capable of generating CO₂ gas within a satellite container until a user decides to allow the CO₂ gas or material capable of generating CO₂ gas to be released and moved into an inner region of platelet container component 202.

In some cases, a satellite container can be designed to house blood components such as red blood cells or plasma. For example, satellite container 206 can be designed to house red blood cells, and satellite container 208 can be designed to house plasma.

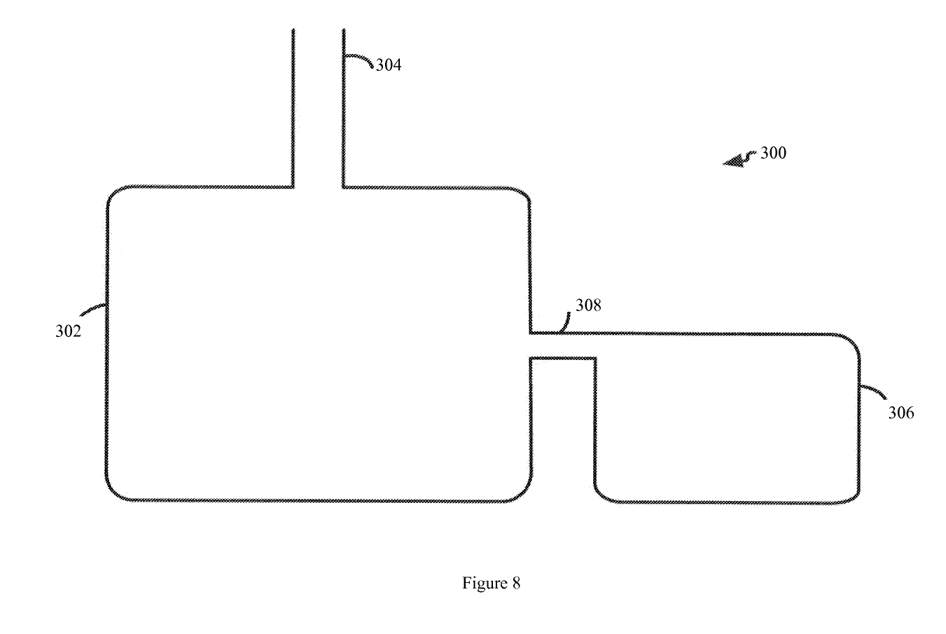

With reference to FIG. 8, platelet container system 300 can include a platelet container component 302 and an inlet port 304 configured to allow a platelet preparation to be inserted into platelet container component 302. Platelet container system 300 can include one satellite container (e.g., satellite container 306). Satellite container 306 can be fluidly connected to an inner region of platelet container component 302 via a channel 308 (e.g., a tube). Satellite container 306 can contain CO₂ gas or material capable of generating CO₂ gas. In some cases, platelet container system 300 can include a valve or membrane configured to retain CO₂ gas or material capable of generating CO₂ gas within satellite container 306 until a user decides to allow the CO₂ gas or material capable of generating CO₂ gas to be released and moved into an inner region of platelet container component 302.

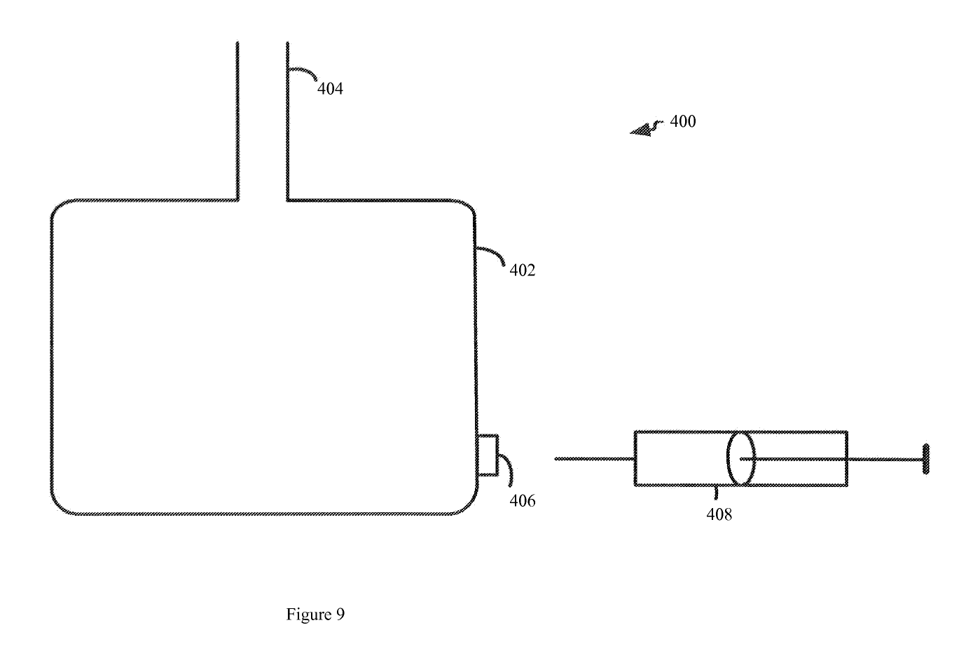

In some cases, a platelet container component provided herein can include an injection port. For example, with reference to FIG. 9, a platelet container device 400 can include a platelet container component 402 and an inlet port 404 configured to allow a platelet preparation to be inserted into platelet container component 402. Platelet container device 400 can include an injection port 406. Injection port 406 can be configured to allow a needle (e.g., of a syringe 408) to be inserted into an inner region of platelet container component 402. The syringe can be used to deliver CO₂ gas or material capable of generating CO₂ gas into an inner region of platelet container component 402 in a sterile manner. In some cases, injection port 406 can be configured to seal upon removal of an inserted needle.

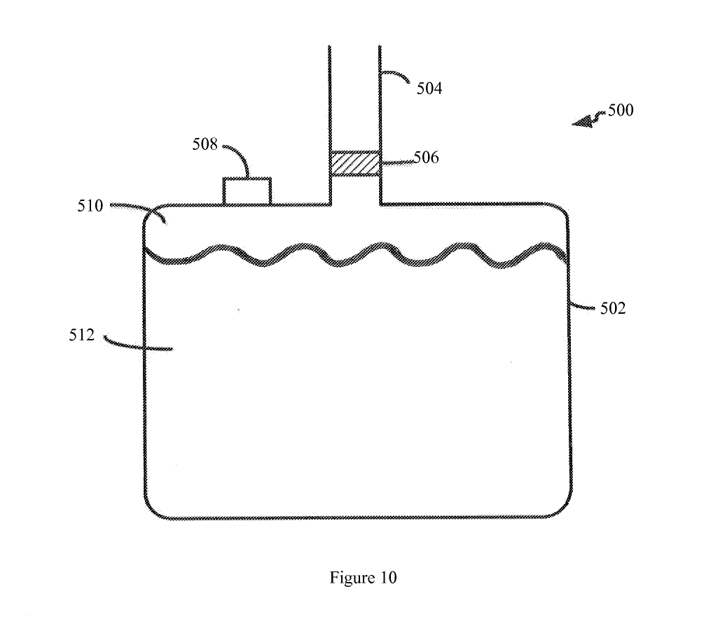

In some cases, a platelet container component provided herein can include an exhaust valve. For example, with reference to FIG. 10, a platelet container device 500 can include a platelet container component 502 and an inlet port 504 configured to allow a platelet preparation 512 to be inserted into platelet container component 502. Platelet container device 500 can include an exhaust valve 508. Exhaust valve 508 can be configured to allow a user to remove gas 510 present within an inner region of platelet container component 502 in a sterile and sealable manner.

In some cases, a platelet container component provided herein can include an inlet port 504 having a valve. For example, with reference to FIG. 10, inlet port 504 can be configured to have valve 506. Valve 506 can be configured to have open and closed configurations. When in an open configuration, valve 506 can allow fluids and gases to pass into and out of an inner region of platelet container component 502. When in a closed configuration, valve 506 can prevent the contents within an inner region of platelet container component 502 from exiting that region via inlet port 504.

In some cases, a bag system can include a series of bags to facilitate the separation of blood into components. The blood can be first collected as whole blood (e.g., typically around 500 cubic centimeters) in a large bag, and other bags can receive the separated products during processing. In some cases, one of the bags not used in the initial collection of whole blood can be loaded with CO₂ for subsequent evacuation and mixing with the platelets. In some cases, a relatively small amount (e.g., 50-100 cubic centimeters) of CO₂ can be used to saturate the platelets completely.

In some cases, a bubble-like wrap-like reservoir with CO₂ can be incorporated into a platelet bag, and a simple one-way valve can be configured to permit evacuation of CO₂ with gentle squeezing into the remaining platelets. In some cases, a sterile one-way valve can be used to permit the rapid dosing of CO₂ from a traditional gas tank or other source. This can be configured in a manner that injects a known amount of CO₂ in the bag. Due to the nature of gas in closed spaces and equilibration characteristics of CO₂ and platelets, minimal mixing may be needed. In some cases, dry ice or another chemical reservoir that includes a phase change from solid to gas can be placed in the platelet bag to liberate CO₂.

In some cases, gases such as hydrogen sulfide (i.e., H₂S), HS, or isofluoroane can be used in addition to CO₂ or in place of CO₂. For example, hydrogen sulfide can be used to store red blood cells or platelets as described herein using hydrogen sulfide in place of CO₂.

In some cases, red blood cells or platelets can be stored within a gas-impermeable container (e.g., a gas-impermeable bag such as a CO₂ gas impermeable bag made of PET) in the presence of CO₂ or another gas as described herein. In some cases, red blood cells or platelets can be stored within a gas-permeable container (e.g., a gas-permeable bag such as a current PL732 platelet bag) that can be stored within another container (e.g., a sealed cabinet) designed to have a particular amount of CO₂ or another gas such that the gas (e.g., CO₂ or another gas) can diffuse into the gas-permeable container housing the red blood cells or platelets.

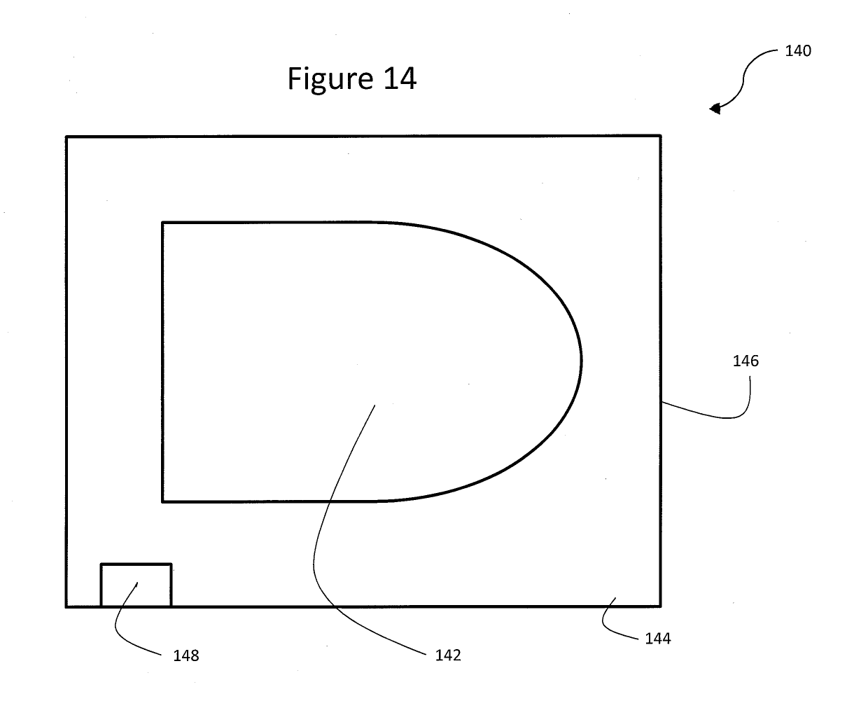

For example, with reference to FIG. 14, red blood cells or platelets can be stored within a gas-permeable container 142 (e.g., a gas-permeable bag such as a current PL732 platelet bag) that can be inserted into a larger container 144. Larger container 144 can be a gas-impermeable container (e.g., a gas-impermeable bag such as a CO₂ gas impermeable bag made of PET). Larger container 144 can include one or more openings or edges that allow a user to insert gas-permeable container 142 into larger container 144. In some cases, such one or more openings can be configured to allow a user to seal larger container 144 such that larger container 144 is gas-impermeable. For example, larger container 144 can include a sealable edge 146 that can be opened and closed to allow a user to insert gas-permeable container 142 into larger container 144 or to remove gas-permeable container 142 from larger container 144. In some cases, larger container 144 can be configured to contain one or more than one gas-permeable container(s) 142. For example, larger container 144 can be configured to contain two, three, four, five, six, seven, eight, nine, ten, or more gas-permeable containers. In some cases, larger container 144 can include an injection port 148 configured to allow a user to inject CO₂ gas or another gas into larger container 144.

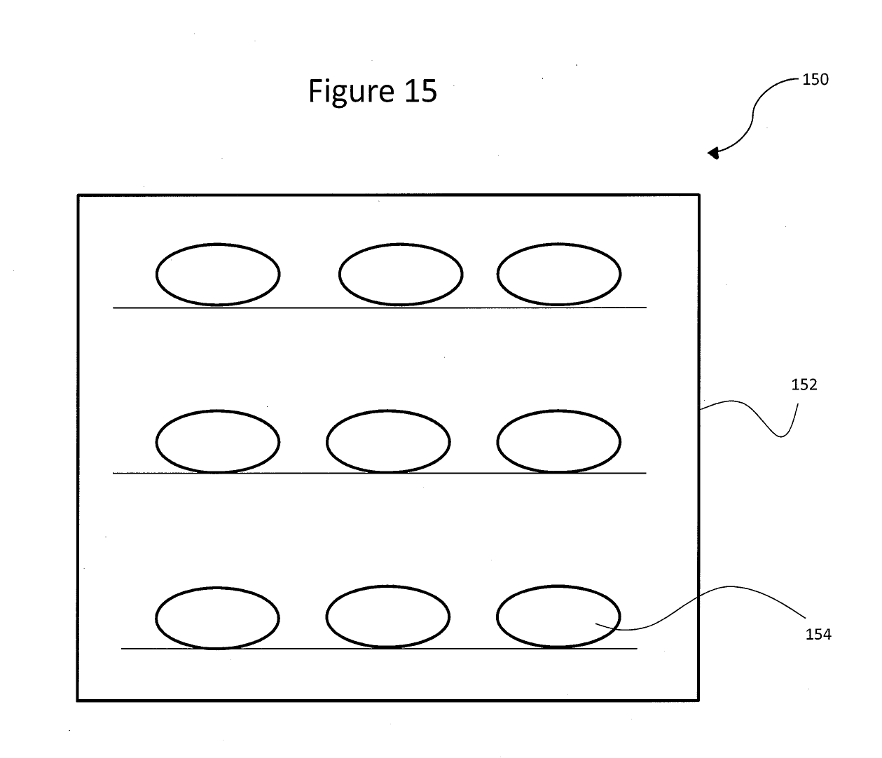

In another example, with reference to FIG. 15, red blood cells or platelets can be stored within a gas-permeable container 154 (e.g., a gas-permeable bag such as a current PL732 platelet bag) that can be inserted into a cabinet 152. Cabinet 152 can be a gas-impermeable cabinet or chamber. Cabinet 152 can include a door that allows a user to insert gas-permeable container(s) 154 into cabinet 152 or remove gas-permeable container(s) 154 from cabinet 152. In some cases, such a door can be configured to allow a user to seal cabinet 152 such that cabinet 152 is gas-impermeable. In some cases, cabinet 152 can be configured to contain one or more than one gas-permeable container(s) 154. For example, cabinet 152 can be configured to contain two, three, four, five, six, seven, eight, nine, ten, or more gas-permeable containers. In some cases, cabinet 152 can be configured to inject CO₂ gas or another gas into the inner region of cabinet 152. In some cases, cabinet 152 can be configured to have a temperature-controlled environment within the cabinet. For example, cabinet 152 can include a temperature gauge and a cooling system such that a user can set an inner compartment of cabinet 152 to a particular temperature (e.g., between 10° C. and −5° C., between 5° C. and −1° C., between 4° C. and −2° C., between 1° C. and −1° C., or between 10° C. and 4° C.). In some cases, cabinet 152 can include CO₂ sensor to allow for the constant monitoring and control of CO₂ levels.

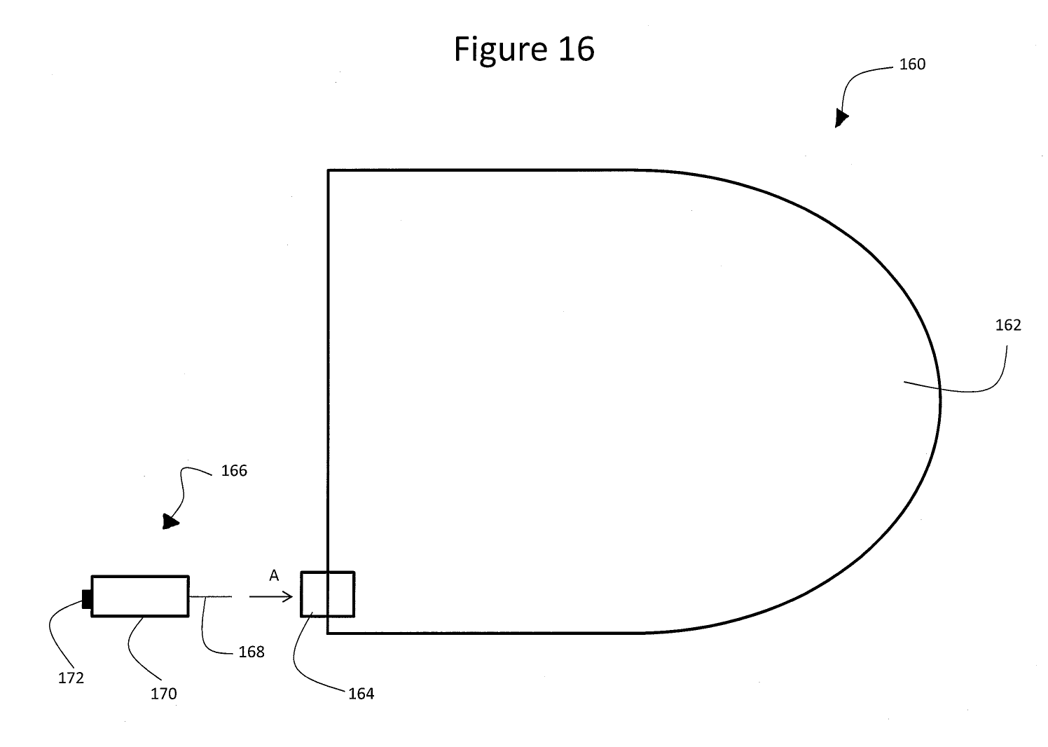

With reference to FIG. 16, red blood cells or platelets can be stored within a container 162. Container 162 can include an injection port 164 configured to allow a user to inject CO₂ gas or another gas into container 162. In some cases, a cartridge 166 having a needle portion 168 and a housing portion 170 can be pre-filled with CO₂ or another gas under pressure. In some cases, a luer lock fitting or other fitting can be used in place of needle portion 168. Cartridge 166 can include an actuator button or switch 172 configured to release the CO₂ or other gas. During use, a user can attach cartridge 166 to container 162 and press actuator button or switch 172 to inject CO₂ or another gas into container 162. The CO₂ or other gas can be injected pre- or post-blood collection. Prior to use or release for use, a red blood cell or platelet preparation provided herein can be de-gassed or treated in a manner to remove a CO₂ gas or another gas. For example, a red blood cell or platelet preparation provided herein can be swirled over or in the presence of atmospheric air or O₂ (e.g., 100% O₂) until the pH of the preparation is greater than about 7.2 (e.g., until the pH of the preparation returns to a pH that is greater than pH 7.2). In some cases, room air or 100% oxygen can be used to reverse the pH to a level where the pH is above about 7.0 in around, for example, 5 to 20 minutes (e.g., about 10 minutes).

The invention will be further described in the following examples, which do not limit the scope of the invention described in the claims.

### EXAMPLES

### Example 1

### Storing Red Blood Cells in the Presence of CO₂

Red blood cells were collected and processed using standard techniques and stored in standard blood bags. Prior to collecting the red blood cells, an empty blood bag containing anti-coagulated citrate phosphate dextrose with adenine (AS1) was flushed with CO₂ for one minute. The collection bag was then filled with blood from a donor. Next, 50 cubic centimeters of CO₂ were added to the bag via the sterile docking port. The bag was gently inverted for 30 seconds and then placed upright. Using an empty syringe, the 50 cubic centimeters of air/CO₂ was removed from the bag through the sterile docking port. The controls were red blood cell preparations placed in blood bags not flushed with CO₂ and not exposed to 50 cubic centimeters of CO₂.

The control and CO₂ treated red blood cell preparations were tested for pCO₂ (or pO₂) levels, lactate levels, glucose levels, and pH levels at days 0, 14, and 21 (Table 1). Storage with CO₂ resulted in a pCO₂ of >500 mmHg in the bag and a fall in pH to the 6.2 to 6.3 range (Table 1). This fall in pH is reversible, and pH levels can return to normal values of about 7.0 when normal pCO₂ levels (e.g., 40-60 mmHg) are re-established. Storage with CO₂ also resulted in an inhibition of lactate formation and glucose consumption as compared to the levels of lactate formation and glucose consumption exhibited in controls (Table 1).

TABLE 1
pCO₂ levels, lactate levels, glucose levels, and pH levels for red blood cell
preparations exposed or not exposed to CO₂.

| | pCO₂ | | Lactate | | Glucose | | | |
|---|---|---|---|---|---|---|---|---|
| | (mmHg) | | (μmol/mL) | | (mg/dL) | | pH | |
| | CO₂ | Con- | CO₂ | Con- | CO₂ | Con- | CO₂ | Con- |
| Days | Treated | trol | Treated | trol | Treated | trol | Treated | trol |
| 0 | 527 | 53 | 1.2 | 1.2 | 571 | 571 | 6.2 | 7 |
| 14 | 528 | 56 | 5 | 9.4 | 503 | 419 | 6.2 | 7 |
| 21 | 501 | 49 | 5.3 | 17.82 | 478 | 187 | 6.2 | 6.8 |

These results demonstrate that storing red blood cells under conditions of greater than 100 mmHg of pCO₂ (e.g., greater than 200 mmHg of pCO₂, greater than 300 mmHg of pCO₂, greater than 400 mmHg of pCO₂, or greater than 500 mmHg of pCO₂) can inhibit glycolysis, suppress metabolism, and reduce oxidative stress, thereby prolonging the usable lifespan of the red blood cell preparation.

### Example 2

### Storing Platelets in the Presence of CO₂

Fresh human whole blood was drawn into 3.2% sodium citrate, and platelet-rich plasma (PRP) was prepared. Control citrated PRP was stored at room temperature in a polypropylene tube. CO₂ was exposed to the air/fluid interface of PRP using a 99.9% CO₂ tank. CO₂ was administered for approximately 60 seconds during which time the pH dropped from an average value of 7.4 to 6.4.

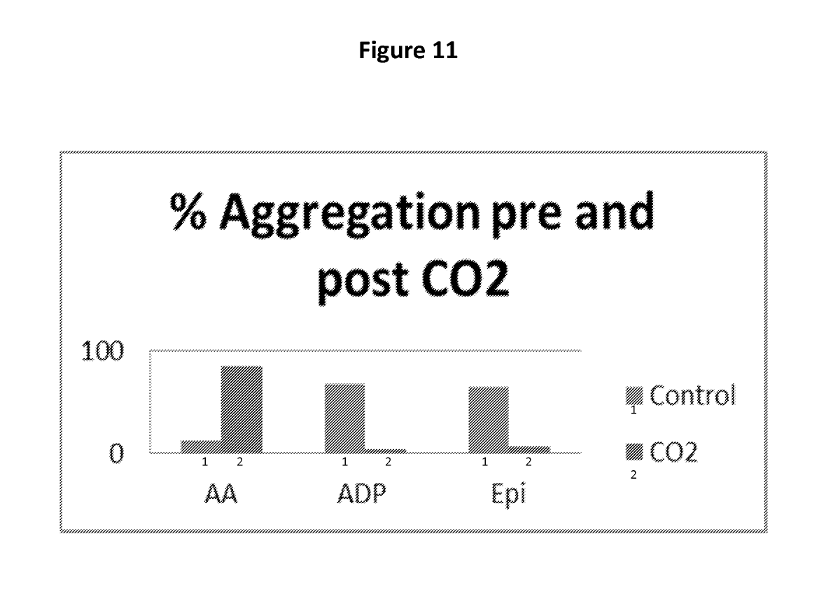

Using a Chronolog 700 platelet aggregometer, platelet aggregation was performed pre- and post-CO₂ to ADP, AA, and Epi. Platelet aggregometry was carried out as described elsewhere (Born and Cross, J. Physiol., 168:178-195 (1963)). The assay was performed at 37° C. with a sample stir speed of 1200 r.p.m. Each sample consisted of 450 μL platelet rich plasma. The results are provided in FIG. 11.

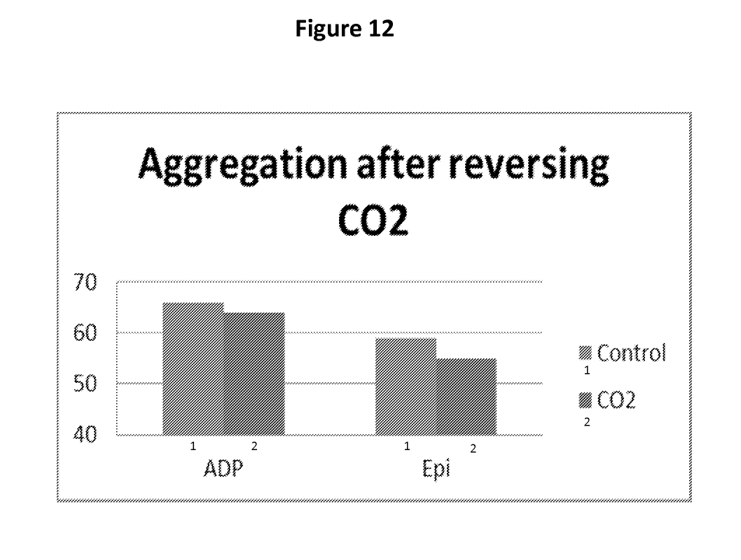

Aggregation was again recorded after reversing the CO₂ effect by adding atmospheric air into the PRP until the pH reached 7.2 (FIG. 12).

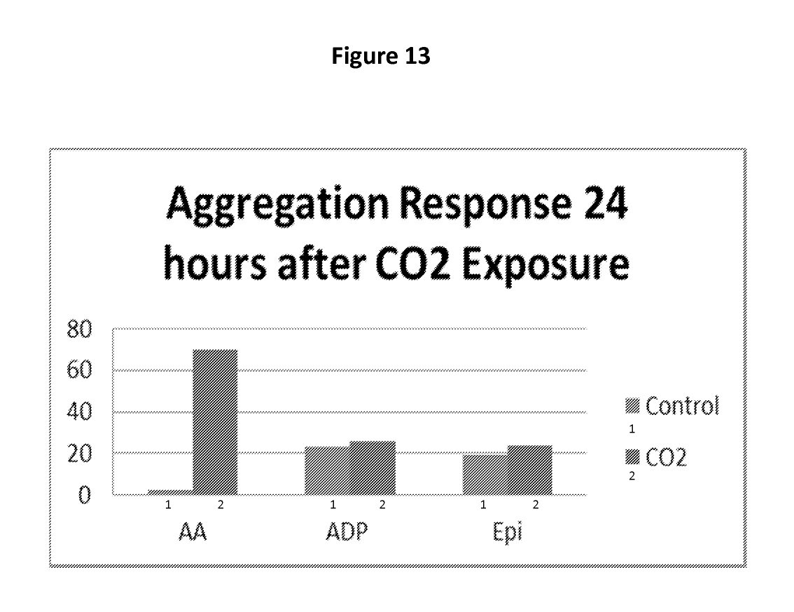

In addition, platelet aggregation was measured after 24 hours storage at room temperature for both the control and CO₂ treated group (FIG. 13).

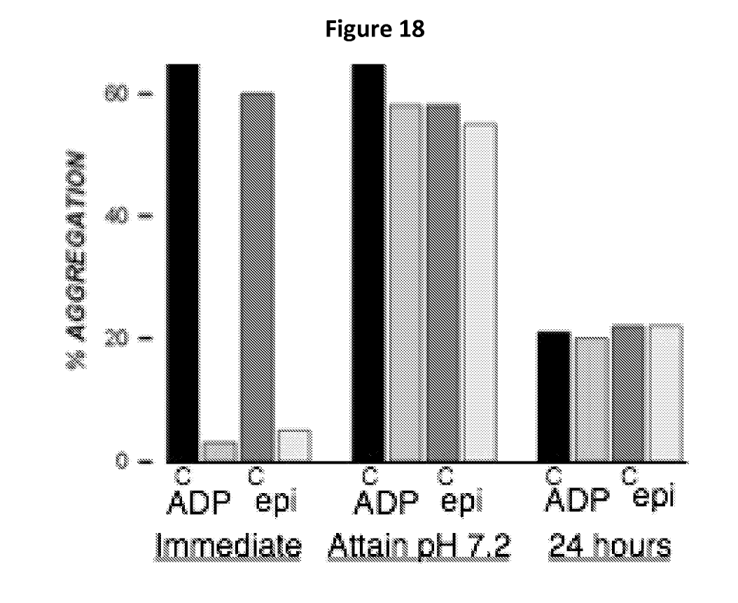

In another study, the impact of CO₂ on platelet function was explored by equilibrating platelet-rich plasma (PRP) with 99% CO₂. The PRP was prepared by low-speed centrifugation of blood anticoagulated with 3.2% sodium citrate and kept in capped polypropylene tubes. Platelet function was measured by optical aggregometry with ADP and epinephrine as triggers. Equilibration with CO₂ under a positive flow of gas was monitored by the fall in pH from 7.2 to 6.4, which typically occurs within 60 seconds. Degassing of CO₂ by a flow of room air over the PRP was verified by the return to pH 7.2. Immediately after removal of the PRP from the CO₂ atmosphere, aggregation to ADP and epinephrine was suppressed (FIG. 18, first set). When the samples had returned to pH 7.2, aggregation to both triggers was restored (FIG. 18, center set). After 24 hours under CO₂, aggregation was equivalent to that of controls (FIG. 18, third set).

### ATP Secretion Response Pre and Post CO₂

ATP secretion was performed using a Zylux luminometer where the secretion was induced using 1 μM thrombin. In this test, the ATP secretion rate and total ATP for the control group decreased significantly (to less than 15%) after 24 hours storage at room temperature. However, the CO₂ treated platelets had approximately 80% of baseline secretion after 24 hours storage. The CO₂ did not need to be reversed in order for the secretion response to proceed.

### Bacterial Growth Inhibition Post CO₂

Clinical isolates of Bacillus cereus and Pseudomonas aeruginosa were spiked into control and CO₂ treated platelets at a concentration of 10 CFU (i.e., 10 bacteria total, not 10 CFU/mL). The platelets were stored for approximately 30 hours at room temperature on a platelet agitator. After 30 hours, quantitative culture was performed on serial dilutions of the platelet sample on 5% sheep blood agar plates.

Bacillus cereus grew to over x10e5 CFU/mL for the control PRP, and no growth was observed for the sample containing Bacillus cereus exposed to CO₂. Pseudomonas aeruginosa grew to x10e3 CFU/mL for the control PRP, whereas no growth occurred on the Psudomonas aeruginosa spiked PRP that was exposed to CO₂.

### Example 3

### Storing Platelets

Apheresis donor platelets, collected in PL732 citrate-dextrose-phosphate (CPD) platelet bags, using the Baxter/Fenwal Amicus Apheresis Instrumentation, are obtained in a routine manner following a transfusion protocol. These apheresis donor platelets undergo functional analyses in the presence and absence of CO₂ treatment. The following functional parameters are assessed on days 1 (day of collection) and 5 of storage: platelet activation, as indicated by an increase in the level of P-selectin expression; adenine nucleotide content, as determined by ATP levels and measured ATP release using bioluminescence; and platelet aggregation to 20 μM ADP, 10 μM epi, 0.4 mM AA, and collagen.

The bags (or segment tubes) of apheresis platelets are obtained from Mayo Transfusion Services. Each bag (or segment) of platelets is subsequently divided into a number of equal aliquots. The aliquots are stored on a platelet agitator at room temperature in sterile containers. Within 2 hours of obtaining the apheresis platelets, a number of samples are treated with CO₂, and a number of samples are used as controls. All samples are assayed for in vitro functional parameters.

### Flow Cytometry for P-Selectin Expression

Platelet activation is assessed using flow cytometry by monitoring P-selectin expression. Twenty μL of PC was diluted (100-fold) into a buffer containing 1 μm hirudin. The diluted blood samples are centrifuged for 10 minutes at 1000 g. Supernatants are discarded, and the cell pellets are resuspended in buffer. Diluted whole-blood samples (100 μL) are activated with human thrombin (10.0 nM for 10 minutes; Haematologic Technologies, Essex Junction, Vt.) and are stained simultaneously for glycoprotein β3 and P-selectin (30 minutes) using mouse monoclonal immunoglobulin G (IgG) against P-selectin (CD62) conjugated to phycoerythrin (PE) and mouse monoclonal IgG against glycoprotein IIbIIIa (CD61) conjugated to fluorescein isothiocyanate (FITC) (Becton-Dickinson, San Jose, Calif.). Each sample is fixed by the addition of 100 μL of a 1:20 dilution of formalin for 30 minutes and then is neutralized with 0.2 M Tris, to a pH of 8.0. Samples are then diluted down to 2 mL for analysis. Samples are analyzed using a Partec (Muenster, Germany) CA3 flow cytometer.

### Bioluminescence for ATP Secretion

Apheresis platelets, with and without CO2 exposure, are assayed for the rate of dense body ATP secretion and total platelet ATP content using a custom designed luminometer. Platelet ATP secretion is measured by adding luciferase (1 mg/mL) and luciferin (10 μg/mL) to each sample (Owen et al., Biochemistry, 34:9277-9281 (1995) and Kahn et al., Nature, 394:690-694 (1998)). The luminescence generated by platelet release of ATP is compared with that of an ATP standard. For each assay, 40 mL of platelets diluted 1:1000 is mixed with 10 mL of luciferase reagent (0.5 mg/mL luciferase, 1.4 mg/mL luciferin) (Sigma Chemical Co., St Louis, Mo.) and is placed in a photomultiplier tube compartment. Diluted platelets are activated with 50 μL of 10.0 nM human alpha-thrombin. Data are acquired using an OLIS (OLIS, Bogart, Ga.) interface and software.

Response is measured as rate of ATP secretion by plotting the slope of the secretion curve vs. time. Total ATP content is measured as the amount of ATP released after lysis of cells using a detergent (50 μL of a 1:100 dilution of Triton-X-100). Without the ability to aggregate, platelets are unable to form effective haemostatic plugs, which are necessary to control active and chronic bleeding. The relative response of platelets to various agonists is routinely measured photometrically (Chronolog Corp., Havertown, Pa.). Each platelet sample is challenged with an agonist to promote aggregation, as evidenced by clumping. Once a platelet aggregate forms, more light is able to pass through the sample. The amount of light passing though is measured by photometric analysis. An increase in the level of platelet aggregation is manifest as an increase in light emission, with its relative intensity recorded by a turbidometer. Weak aggregation allows relatively less light to pass through, and no aggregation allows little or no light to pass above the baseline level.

Data analyses of the results of all samples assessing functional parameters are reported as percentage change, mean, or observed change. When appropriate, the data analysis for each assessed parameter between both groups (experimental preservative solution-treated vs. untreated) is performed using a paired Student's t-test.

### Bacterial Growth by Traditional Culture

The impact of CO₂ on apheresis collected platelets is evaluated for the following 15 microorganisms: Bacillus cereus, Bacillus subtilis, Clostridium perfringens, Corynebacterium species, Echerichia coli, Enterobacter cloacae, Klebsiella oxytoca, Propionibacterium acnes, Pseudomonas aeruginosa, Serratia marcescens, Staphylococcus aureus, Staphylococcus epidermidis, Streptococcus pyogenes, viridans group Streptococcus species, and Candida albicans. For each isolate, colonies are inoculated from the 5% sheep blood agar plate into Tryptic Soy Broth (TSB) at 5×10⁷ to 1×10⁸ CFU/mL (0.5-1.0 McFarland standard). A 1:100 dilution is made into 20 mL TSB for inoculating into platelets (˜10⁶ CFU/mL) to yield a bacteria stock.

Leukoreduced Apheresis Platelet (LRAP) units obtained from Mayo Transfusion Services blood bank are inoculated with bacteria at an approximate concentration of 100 bacteria. A 50 mL LRAP unit aliquot will be spiked with 10 microliters of a single organism bacterial stock to yield a final concentration of 1-10 CFU/mL. Initial concentration of spike and grow inoculums are quantified via traditional culture. Additionally, an uninoculated LRAP aliquot is preserved for bacterial growth determination. Inoculated LRAP aliquots are maintained in a platelet agitator at 22-25° C. for 24 hours. Following the incubation period, 1 mL samples of each spiked LRAP aliquot are collected, and the remaining spiked sample is left in incubation for an additional 24 hours to screen for slow growing organisms.

### Example 4

### Inhibition of Bacterial Growth within Platelet Preparations Stored Under CO₇ Conditions

Outdated apheresis platelet unit (day 6) was obtained from the Mayo Clinic components laboratory. About half of the platelet unit was perfused with 99% CO₂ gas for 10 minutes with mixing. Control samples without CO₂ exposure and CO₂-treated samples of 10 mL volume were inoculated with clinical isolates of eight different organisms. Control and CO₂ samples were spiked with identical bacterial loads. Quantitative culture using 5% sheep blood agar plates was conducted immediately following inoculation (0 hours) and at 24 and 48 hour time points. Time 0 hour inoculation load was selected to compensate for observed “spike and die” in outdated apheresis platelet units. The time 0 hour value represented the value of the initial spike.

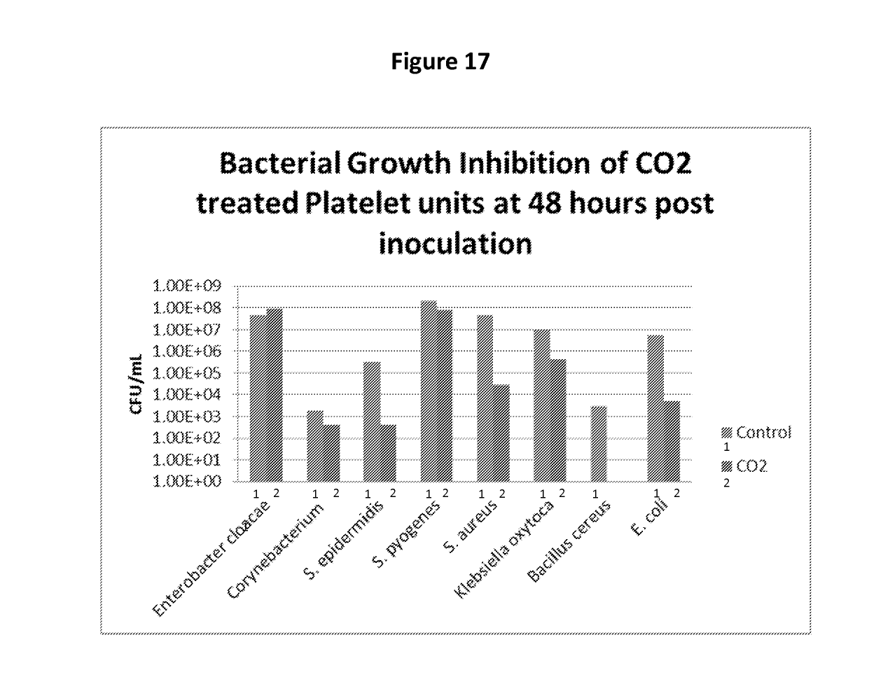

Bacterial growth was reduced for those samples exposed to 99% CO₂ gas for 10 minutes (Table 2 and FIG. 17).

TABLE 2

| | Control | | | CO₂ | | |
|---|---|---|---|---|---|---|
| Organism | 0 hours | 24 hours | 48 hours | 0 hours | 24 hours | 48 hours |
| S. epidermidis | 5.10E+02 | 1.41E+03 | 3.30E+05 | 6.10E+02 | 3.30E+02 | 4.20E+02 |
| S. pyogenes | 1.67E+03 | 1.12E+06 | 2.10E+08 | 1.55E+03 | 1.20E+05 | 8.00E+07 |
| S. aureus | 1.14E+03 | 3.40E+05 | 4.80E+07 | 1.17E+03 | 3.20E+03 | 2.70E+04 |
| Klebsiella | 9.80E+02 | 6.60E+05 | 1.00E+07 | 8.10E+02 | 1.15E+04 | 4.30E+05 |
| oxytoca | | | | | | |
| Bacillus cereus | 5.00E+01 | 8.00E+01 | 2.90E+03 | 1.00E+01 | no | no |
| | | | | | growth | growth |
| E. coli | 4.00E+01 | 2.70E+03 | 5.50E+06 | 1.00E+01 | 1.80E+02 | 5.20E+03 |
| Enterobacter | 1.10E+03 | 9.90E+05 | 4.70E+07 | 1.22E+03 | 1.20E+06 | 8.60E+07 |
| cloacae | | | | | | |
| Corynebacterium | 3.20E+02 | 2.30E+02 | 1.90E+03 | 7.90E+02 | 2.70E+02 | 4.20E+02 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing the rate of lactate formation or glucose consumption by stored red blood cells of a red blood cell preparation, wherein said method comprises exposing said red blood cell preparation to CO₂ gas under conditions wherein the pCO₂ level of said red blood cell preparation is greater than 100 mmHg of pCO₂.

2. The method of claim 1, wherein said method comprises exposing said red blood cell preparation to CO₂ gas under conditions wherein the pCO₂ level of said red blood cell preparation is greater than 200 mmHg of pCO₂.

3-5. (canceled)

6. The method of claim 1, wherein said method comprises exposing said red blood cell preparation to CO₂ gas under conditions wherein the pCO₂ level of said red blood cell preparation is between 200 and 600 mmHg of pCO₂.

7. (canceled)

8. The method of claim 1, wherein said method comprises reducing said rate of lactate formation to a level that results in less than 7.5 μmol/mL of lactate being formed after 14 days of storage.

9. The method of claim 1, wherein said method comprises reducing said rate of lactate formation to a level that results in less than 10 μmol/mL of lactate being formed after 21 days of storage.

10. The method of claim 1, wherein said method comprises reducing said rate of glucose consumption to a level that results in greater than 450 mg/dL of glucose being present after 14 days of storage.

11. The method of claim 1, wherein said method comprises reducing said rate of glucose consumption to a level that results in greater than 200 mg/dL of glucose being present after 21 days of storage.

12-13. (canceled)

14. The method of claim 1, wherein the pH of said red blood cell preparation is less than 6.8 after 14 days of storage.

15-16. (canceled)

17. The method of claim 1, wherein the pH of said red blood cell preparation is less than 6.6 after 21 days of storage.

18-34. (canceled)

35. A blood container comprising an inlet port configured to allow blood to be inserted into said blood container and a capsule comprising CO₂ gas or material capable of generating CO₂ gas, wherein said capsule is located in a position that allows said CO₂ gas or said material to be delivered to an inner region of said container.

36. The blood container of claim 35, wherein said capsule is located within said inner region of said container.

37-39. (canceled)

40. The blood container of claim 35, wherein said blood container comprises an exhaust valve configured to allow gas to be removed from said inner region of said blood container.

41. A blood container system comprising a blood container having an inlet port configured to allow blood to be inserted into said blood container and a satellite container comprising CO₂ gas or material capable of generating CO₂ gas, wherein said satellite container is configured to be in fluid communication with an inner region of said blood container.

42. The system of claim 41, wherein said system comprises a valve configured to retain said CO₂ gas or material within said satellite container.

43. The system of claim 42, wherein said valve is capable of being opened to allow said CO₂ gas or material to exit said satellite container into said inner region of said blood container.

44. The system of claim 41, wherein said system comprises a membrane configured to retain said CO₂ gas or material within said satellite container.

45. The system of claim 44, wherein said membrane is capable of being broken to allow said CO₂ gas or material to exit said satellite container into said inner region of said blood container.

46. The system of claim 41, wherein said blood container comprises an exhaust valve configured to allow gas to be removed from said inner region of said blood container.

47-48. (canceled)

49. A platelet container comprising an inlet port configured to allow platelets to be inserted into said platelet container and a capsule comprising CO₂ gas or material capable of generating CO₂ gas, wherein said capsule is located in a position that allows said CO₂ gas or said material to be delivered to an inner region of said container.

50. (canceled)

51. A platelet container comprising an inlet port configured to allow platelets to be inserted into said platelet container and an injection port configured to allow sterile insertion of a needle for delivering CO₂ gas or material capable of generating CO₂ gas into an inner region of said container.
